(19) United States
(12) Patent Application Publication
(10) Pub. No.: US 2022/0122714 A1
(43) Pub. Date: Apr. 21, 2022

(54) Real Time Intervention to a User's Mood

(71) Applicant: Susanne Moore, Birmingham, AL (US)

(72) Inventor: Susanne Moore, Birmingham, AL (US)

(21) Appl. No.: 16/459,546

(22) Filed: Jul. 1, 2019

(51) Int. Cl. G16H 20/70; H04W 4/12; G06V 40/20; G06V 40/16

(52) U.S. Cl. CPC G16H 20/70; G06V 40/16; G06V 40/20; H04W 4/12

(57) ABSTRACT

A computer readable medium, program and method configured to deliver instructions to cause a computing device to respond to a user's mood input with actions to provide a user support and emotional relief and improve a user's thought patterns and behaviors. A computer readable medium, program and method for responding to and modifying a user entered mood and mood level intensity, the program and method capable of auto-notifying at least one user specified support system member and generating a customized response in response to the mood and mood level intensity entry, the response consisting of personalized and gamified on demand action steps from which the user may select.

### CROSS REFERENCE TO RELATED APPLICATIONS

This application claims the benefit of provisional application 62/694,483 filed Jul. 6, 2018 by the same inventor and the same is incorporated in its entirety herein.

### STATEMENT REGARDING FEDERALLY SPONSORED RESEARCH OR DEVELOPMENT

Not Applicable

### NAMES OF PARTIES TO JOINT RESEARCH AGREEMENT

Not Applicable

### REFERENCE TO SEQUENCE LISTING

Not Applicable

### DESCRIPTION OF ATTACHED APPENDIX

Not Applicable

### BACKGROUND OF THE INVENTION

### 1. Field Of The Invention

The present disclosure relates to a computer readable medium, program and method for responding to and modifying a user entered mood and mood level intensity to provide on-demand emotional relief and long-term thought pattern and behavior modification.

### 2. Description of Related Art

Mobile software applications are common to support, in an on-demand manner, the needs of an end user. It is becomingly increasingly common to find meditation mobile applications, and even on-demand online counseling services. Nonetheless, there is still a certain stigma related to seeking mental health resources as well as a level of inconvenience and time-delay involved. In addition, when a person is in the throes of anxiety or depression, it is often difficult to think clearly enough to make decisions about what course of action to take. It is also difficult to muster the motivation to take needed action. A need exists for an intuitive and engaging system and method to help a user receive on demand relief, ongoing support and consistently improve emotional well-being.

### SUMMARY OF THE INVENTION

A computer readable medium, program and method for responding to a user's moods to intervene in real time to improve emotional well-being includes a mobile or stationary software application and server capable of execution on and with a computing device such that the application captures and stores data and generates a response to the user. The computing device may be a computer, mobile smartphone, smart television, media player or mobile tablet computer. The application, when activated, has stored instructions causing it to execute the steps of capturing the user's input profile and mood information and auto-notifying the user's pre-defined support system members. The application is further capable of generating a customized response consisting of personalized and gamified on demand action steps from which the user may choose to perform.

A computer readable medium, program and/or method for capturing and responding to a user's moods to intervene in real time, the medium, program and/or method including a software application capable of operating on a mobile processing device having a display and input capability, the software application capable of connecting with a server capable of storing and sharing data. The application includes a program configured to interact with a processor in a computing device to execute the steps of capturing user input mood and mood intensity data, auto-notify at least one user-specified support system member and generate one or more emotional relief action steps.

A computer readable medium, program and/or method for improving a user's emotional well-being, wherein the software is programmed to respond to user input mood and mood intensity with emotional relief action steps to generate an intuitive on demand experience for a user.

A computer readable medium, program and/or method for improving a user's emotional well-being, wherein should the user enter a pre-programmed specific intensity level for a given mood, the system can automatically notify the support system via short message service (SMS), a multimedia message service center (MMSC), an electronic mail message, an application push notification, social media message text message, other defined communication method.

A computer readable medium, program and/or method for responding to an emotional health state, as represented by input on a mood scale includes configuring a software application to merge personal user input data with a database of action steps to deploy a personalized response of actions steps from which the user can choose to improve the emotional state.

A computer readable medium, program and/or method for personally engaging a user by employing methods of gamification and support system engagement such that they have a desire to repeatedly perform the action steps consistently, thus improving their emotional well-being over time.

A computer readable medium, program and/or method for engaging a user's support system such the support system members are notified when the user needs them most and are guided by the language of the notification on how to most appropriately intervene for the user.

A computer readable medium, program and/or method to track a user's entered mood data and competition of action steps to trend their progress over time and remind the user of past successful interventions.

A computer readable medium, program and/or method for long-term modification and improvement of a user's thought patterns and behaviors, including steps of capturing user input mood and mood intensity data, auto-notifying at least one user-specified support system member and generating one or more emotional growth action steps in a gamified manner such that the user earns badges, levels, or rewards for completing steps.

A computer readable medium, program and/or method for capturing and responding to a user's moods to intervene in real time wherein at least a portion of user input mood and mood intensity data is gathered by facial recognition software.

A computer readable medium, program and/or method as described in accordance with the principles found here in can be used to, over time, improve the user's emotional well-being.

Other objects and advantages of the present invention will become apparent from the following descriptions, taken in connection with the accompanying figures, wherein, by way of illustration and example, an embodiment of the present invention is disclosed.

### BRIEF DESCRIPTION OF THE DRAWINGS

The disclosure will provide details in the following description of preferred embodiments with reference to the following figures wherein:

FIG. 1 illustrates a block diagram of the computing device and components that can be configured to implement different aspects of the various techniques described herein, according to some embodiments.

FIG. 2 is a block/flow diagram showing instructions for capturing and responding to a user's input in accordance with one or more disclosed embodiments; and

FIG. 3 is a screenshot of the application showing a medium, program and method for capturing and responding to a user's mood scale input in accordance with one or more disclosed embodiments; and

FIG. 4 is a screenshot of the application showing a medium, program and method for auto-notifying at least one of a user's pre-defined support system members in accordance with one or more disclosed embodiments; and

FIG. 5 is a screenshot of the application showing a medium, program and method for providing gamified badges and tracking progress in accordance with one or more disclosed embodiments; and

FIG. 6 is a screenshot of the application showing a medium, program and method for generating an action step in accordance with one or more disclosed embodiments; and

FIG. 7 is a block/flow diagram showing a medium, program and method for features in accordance with one or more disclosed embodiments.

### DETAILED DESCRIPTION OF THE PREFERRED EMBODIMENTS

Detailed descriptions of the preferred embodiment are provided herein. It is to be understood, however, that the present disclosure may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner. The presently described principles are directed toward to software applications primarily for mobile devices, although a stationary application for use on a stationary computing device such as a desktop may also be employed. The applications are designed to develop and improve the emotional health and fitness of an individual. The present principles use a stationary or mobile application on a computing device as a tool to provide an on-demand gamified system and method to modify or improve an emotional health state as defined by a user described mood scale. The user may activate the application on demand and may be encouraged by notifications to activate the application daily.

Embodiments in accordance with present principles may take the form of a hardware, software or hardware/software device. In a preferred embodiment, embodiments of the disclosed is a computer application program which includes but is not limited to firmware, resident software, microcode or other acceptable mediums that can accomplish the described principles.

Emotional health illness and symptoms of anxiety, sadness and anger abound. Traditional counseling, coaching and other modalities often lack the on-demand nature needed to respond to a person in crisis or a person wanting to work on their emotional wellness in an on demand and real time manner. In addition, although a person may need the support of family and friends more than ever when they are in deep depression or anxiety, these are often the times they lack the motivation or energy to reach out. This lack of motivation to seek assistance is also why suicide and crisis hotlines are not always as accessible and impactful as we would like them to be. In addition, when an individual is feeling well, they often tend to stop the self-care measures such as counseling, medication that were helping them.

It is scientifically proven that interventions such as human connection, psychological techniques, counseling, coaching, exercise, nutrition and meditation are effective mediums for developing and improving emotional well-being. There are several hurdles to ensuring the consistent application of these principles by a user. One hurdle is a lack of motivation, energy or engagement. Most individuals suffering from depression, anxiety, anger or fear are immersed in their emotions and have difficulty taking the step to seek help and consistently employ the techniques offered. Another hurdle is a lack of insight. Many individuals attribute their negative emotions to the fault of others or to situations and feel powerless to change their state. Even if they have sufficient insight to know they have the power to change their state, they may still lack the knowledge to do so. Books and articles abound but are difficult to digest into easily repeatable habits that could affect real improvement.

In addition, studies have increasingly proven that the most effective indicator of a person's success with recovery from mental health illness or substance abuse, outside of professional medications and therapy, is the presence of connection with a supportive network of family, friends or mentors. Despite this, a user is often hesitant to call or reach out to these persons when they need them the most. The user may not know how to explain their state of mind and needs. The disclosure at hand reveals a system and method for fully engaging a user's support system with minimal and passive action required from the user.

The present disclosure overcomes the described hurdles to overcoming emotional symptoms of anxiety, sadness and anger by a computer medium, system and method that employs an on demand, gamified, user driven personalized experience that breaks the science of improving emotional health into easy to accomplish micro steps, many of which provide immediate relief. In addition, the present disclosure engages the user's support system to ensure that the user maintains connection with a network of persons who care about their well-being. The user can choose steps that are easy or hard, based on their energy level and ability at that point in time. This gives the user defined control over their own growth and needs based on their point in time ability. This aspect of personal control and gamification further engages them to want to continue to use the program in a consistent manner for long term improvement in their emotional growth and well-being.

In addition, the disclosure leverages the benefit of technology to improve a user's experience. Recent studies bring to the light the feasibility of using facial recognition software to identify suicidality and mood changes. In an embodiment, the application may capture mood data from the user not only from the user's categorical selection of mood and mood intensity but also by allowing the application to capture an image of the user's face and analyze the face for indicators of emotional distress.

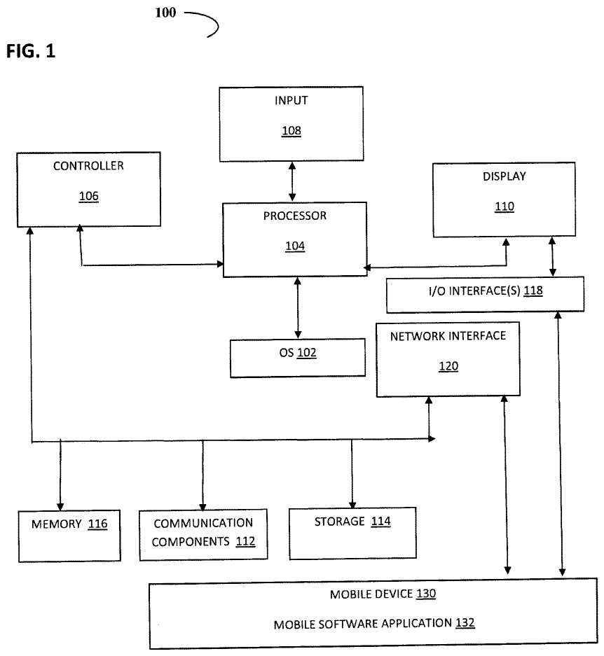

FIG. 1 illustrates a block diagram of the computer readable medium. Specifically, FIG. 1 illustrates a high-level overview of a computing device 100 with an operating system (OS) 102, which, as shown, can include a processor 104, a controller 106, an input 108, a display 110, communication components 112, storage 114 and at least one memory 116. The device would have an I/O interface 118 and a network interface 120. The computing device 100 may be a mobile electronic device 130 operating the instructions of the computer readable medium as a mobile software application 132. According to some embodiments, the processor 104 can be configured to work in conjunction with the memory 116 and the storage 114 to enable the computing device 100 to implement the various techniques set forth in this disclosure. According to some embodiments, the storage 114 can represent a storage that is accessible to the computing device 100, e.g., a hard disk drive, a solid-state drive, a mass storage device, a remote storage device, and the like. The user views a display 110 on the computing device 100 and enters data which the software application 132 then responds to.

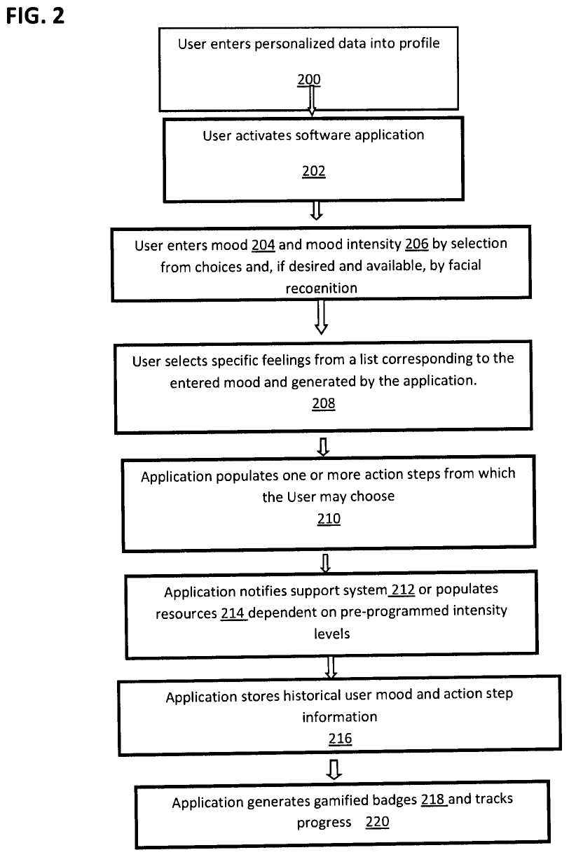

Turning to FIG. 2, in a preferred embodiment of the disclosure, is a block/flow diagram in which like numerals represent the same or similar elements. The user inputs personalized data into the software application profile setup 200. The data in the profile setup 200 may include items such as positive strengths they identify themselves with, their preferred love language or action steps 210 that have helped them in the past. This data is merged with a memory stored database of scientific based action steps in the software application computer program to ensure the user has a personalized experience they can identify with. This encourages long term engagement and also encourages the user to develop more self-awareness and insight. The user is prompted during profile set up 200 to enter contact information for their support system members, for example friends, family, emotional health professionals or mentors. The user then activates the software application 202. The user selects a mood 204 from the mood categories to identify their current emotional state. The user then selects the intensity 206 level of their emotional state. This step encourages self-awareness, allows the software to determine the action steps 210 that should generate and allows the software to determine whether the user's support system should be auto-notified and/or whether mental health crisis resources should populate. The application next asks the user to further define their current feelings, selecting from a list of choices 208. The application next generates an action step 210 that may be customized to their mood and mood intensity entries and that may be personalized based on the data the user entered during profile setup. The user may choose to complete the action step 210 or swipe to the next one. The user may continue this action until they find an action step 210 they wish to and are able to perform. The user driven choice aspect encourages engagement. The action steps 210 may be categorized into five main areas comprising the components of a healthy emotional and emotional system, the areas including nutrition, exercise, connection, awareness and challenges. The mood state and intensity level entered by the user during the mood scale action triggers the software to, based on programmed parameters, auto-alert the user's support system 212 that the user entered during profile set up. The mood state and mood intensity criteria may also, if triggering the programmed parameters, populate hotline and other mental health or emotional health resources 214 that the user may click on for assistance. In addition, dependent on the user entering a specific pre-determined intensity level for a specific pre-determined mood type such as anxiety, sadness, or anger, the application will generate crisis resources such as suicide text lines or local mental health resources.

The software logs the user's mood, mood intensity and the action step 210 chosen and completed 216. The system records the mood scale results and completed action step 210. Following the action step 210 completion, the system can prompt the user to enter efficacy of the step and its effects. Over time, the user can track the progress of their improving emotional well-being. The software generates rewards in the form of gamified badges 218 or other rewards for reinforcement and engagement and tracks progress 220 for trending purposes and long-term historical data. As the user completes each action step 210, the progress scale shows the category filling up with gamified badges 218.

This helps the user learn how each category affects their well-being and how to incorporate action steps from each category into their life. The progress scale and user input data may, in an embodiment, be electronically shared with an emotional health professional with whom the user wishes to have a professional/patient relationship, so that the emotional health professional can easily assess the patient's mood history, interventions and results. This will enable the emotional health professional to more quickly gain a broad clinical picture of the patient. In an embodiment, the user may continue to share the data on an ongoing basis so the emotional health professional can determine the efficacy of any prescribed medications or treatments.

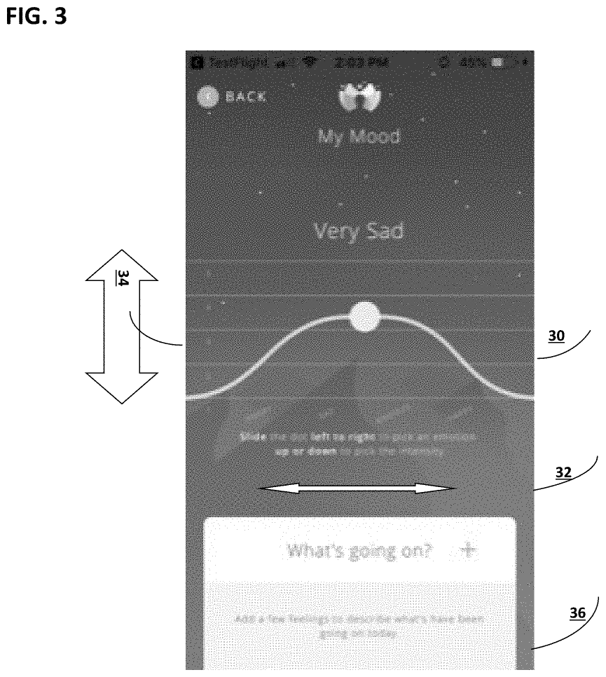

FIG. 3 exhibits a preferred embodiment of the described system and method illustrating a multi-axis mood scale 30 for determining a user's emotional mood state both in terms of type of mood 32 and intensity 34 thereof. Following the user's selection of the mood and mood intensity, in an embodiment, the application next asks the user to further define the specific feelings 46 associated with their mood. All of this information may be captured in the auto-notification to the user's support system member(s).

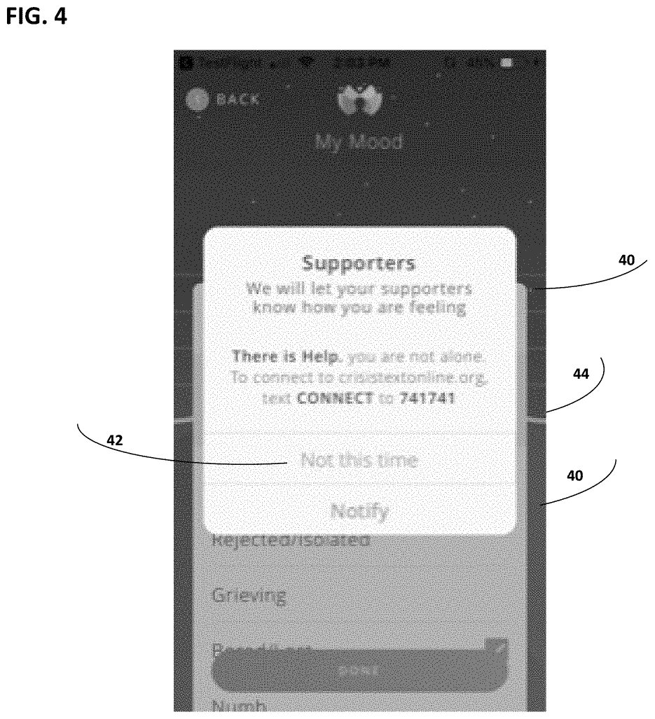

FIG. 4 illustrates the system and method where the application generates auto alert notifications to the user defined support system 40, if appropriate based on the results of the mood scale, as per pre-programmed parameters. The user may decide to override the notification if they so desire 42. The application generates hotlines and resources 44, if appropriate based on the results of the mood scale, as per pre-programmed parameters. The user may then use these resources as desired.

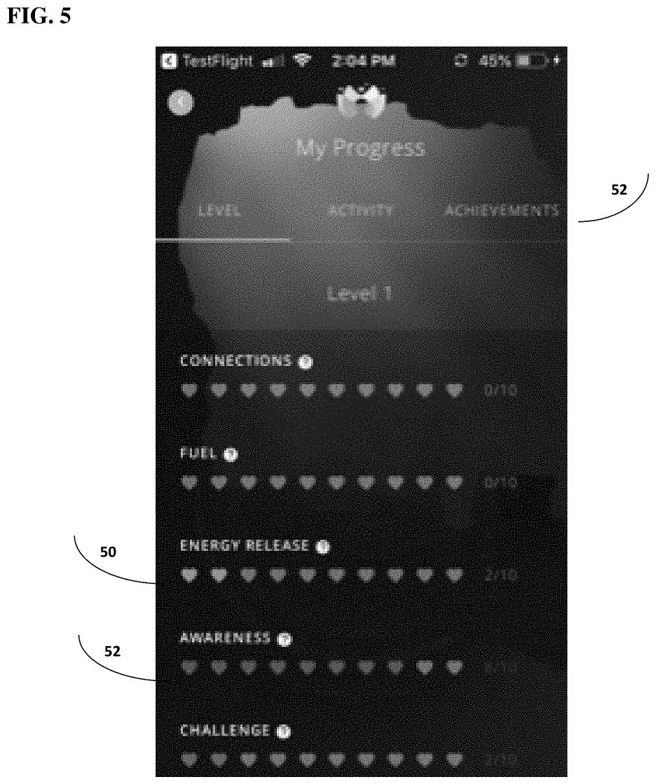

FIG. 5 illustrates the software application generated gamified badges 50 based on the user completing action steps and tracks the progress 52. The data stored on the server or processing device may be shared electronically with professionals as determined by the user.

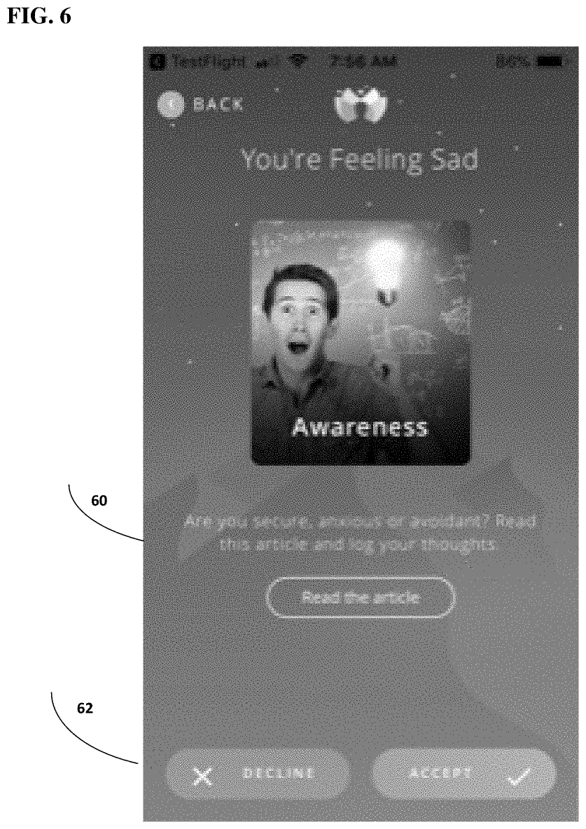

FIG. 6 illustrates an embodiment of the disclosure wherein the application generates an action step 60 in response to the user entered mood information. The user may select the generated step or choose to see additional steps from which to choose 62.

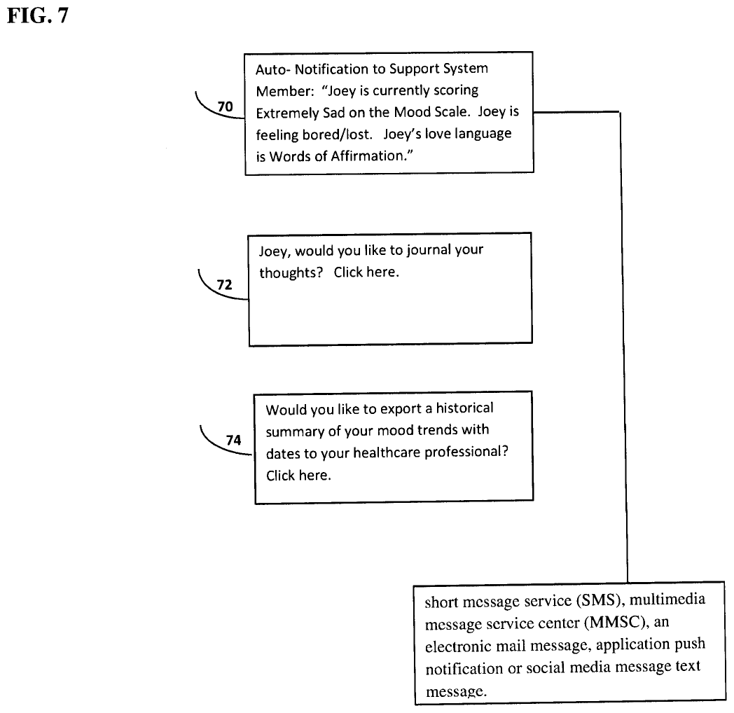

FIG. 7 illustrates an embodiment of the disclosure wherein the application generates an auto-notification 70 to at least one user-defined support system member. Should the user mood scale reach a specific intensity level for a given mood, the auto-notification 70 will alert the support system via short message service (SMS), a multimedia message service center (MMSC), an electronic mail message, an application push notification, social media message text message, other defined communication method. In an embodiment, data related to the user's mood and mood intensity may be captured by facial recognition software. The user may, in an embodiment, choose to enter journal notes 72 corresponding to their mood or action step progress. In an embodiment of the disclosure, the application may include the capacity to export 74 a user's historical mood scale data to a healthcare professional, upon the discretion of the user.

While the disclosure has been described in connection with a preferred embodiment, it is not intended to limit the scope of the disclosure to the particular form set forth, but on the contrary, it is intended to cover such alternatives, modifications, and equivalents as may be included within the spirit and scope of the invention as defined by the appended claims. The various aspects, embodiments, implementations or features of the described embodiments can be used separately or in any combination. Various aspects of the described embodiments can be implemented by software, hardware or a combination of hardware and software. The described embodiments can also be embodied as computer readable code on a computer readable medium. The computer readable medium is any data storage device that can store data which can thereafter be read by a computer system.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of specific embodiments are presented for purposes of illustration and description. They are not intended to be exhaustive or to limit the described embodiments to the precise forms disclosed. It will be apparent to one of ordinary skill in the art that many modifications and variations are possible in view.

The methods and systems disclosed and claimed herein can be made and executed without undue experimentation based on the level of disclosure presented. While the methods and systems have been described in terms of their preferred embodiments, it will be apparent to those skilled in the art that they are not limited to the exact steps described and may vary from such description without departing from the scope and spirit of the invention. The substitutes and modifications employed by one skilled in the art are deemed to fall within the scope of the invention.

What is claimed is:

1. A computer readable medium for responding to a user's moods to intervene in real time which, when executed by a software application on a computing device, cause the device to capture user input mood and mood intensity data, auto-notify at least one user-specified support system member and generate one or more emotional relief action steps for the user to perform.

2. The computer readable medium of claim 1 wherein the mood scale gathers user input data based on mood and intensity of mood, and further wherein the auto-notification is based on the user entered level of intensity.

3. The computer readable medium of claim 1 wherein the software application is a mobile app operating on a mobile electronic device.

4. The computer readable medium of claim 1 wherein the user can override the auto-notification to the at least one user support system members.

5. The computer readable medium of claim 1 wherein the software application auto-generates links to mental health support resources dependent on the input level of mood intensity.

6. The computer readable medium of claim 1 wherein at least a portion of user input mood and mood intensity data is gathered by facial recognition software.

7. The computer readable medium of claim 1 wherein a user's mood scale trends and completion of action steps are recorded and stored.

8. The computer readable medium of claim 1 wherein the auto-notification to the user specified support system members includes at least one of a short message service (SMS), a multimedia message service center (MMSC), an electronic mail message, an application push notification or social media message.

9. The computer readable medium of claim 1 wherein the application has capability for the user to export and share historical data with the user's mental health professional.

10. The computer readable medium of claim 1 wherein said program captures and stores a user's entered notes corresponding to the user's mood and mood intensity data input.

11. A computer program comprising instructions which, when executed by a computing device, cause the device to respond to a user's input with actions to modify a user's thought patterns and behaviors by executing the steps of capturing user input mood and mood intensity data, auto-notifying at least one user-specified support system member and generating one or more emotional growth action steps in a gamified manner such that the user earns rewards for completing steps.

12. The program of claim 11 wherein the computing device is a mobile electronic device operating the instructions in a mobile software application.

13. The medium of claim 11 wherein at least a portion of user input mood and mood intensity data is gathered by facial recognition software.

14. A method enabling a computing device to capture and modify a user's moods, the method comprising: a processing device capable of executing a software application on demand and storing and sharing data, said software application capable of capturing a user entered profile, a user entered mood and a user entered mood level intensity, the application further capable of auto-notifying at least one user specified support system member dependent upon the intensity level of mood entered by the user. The software application further being capable of generating a customized response in response to the mood and mood level intensity entry, the response consisting of personalized and gamified on demand action steps from which the user may select.

15. The method of claim 14 wherein the software application merges user profile entered data with user entered mood and mood intensity to generate a series of on-demand, experience-specific action steps from which the user can choose to execute.

16. The method of claim 14 wherein the user can override the auto-notification to the at least one user support system members.

17. The method of claim 14 wherein the software application auto-generates links to crisis support resources dependent on the user input level of mood intensity.

18. The method of claim 14 wherein a portion of user input mood scale data is gathered by facial recognition software.

19. The method of claim 14 wherein the auto-notification to the user specified support system members includes at least one of a short message service (SMS), a multimedia message service center (MMSC), an electronic mail message, an application push notification or social media message.
